(19) United States
(12) Patent Application Publication
(10) Pub. No.: US 2009/0131692 A1
(43) Pub. Date: May 21, 2009

(54) INTEGRATED PREPARATION AND SEPARATION PROCESS

(75) Inventors: Andre Buijs, Amsterdam (NL); Gerrit Jan Harmsen, Amsterdam (NL); Dominicus Fredericus Mulder, Amsterdam (NL); Anton Pieter Westerink, Amsterdam (NL)

Correspondence Address: SHELL OIL COMPANY, P O BOX 2463, HOUSTON, TX, 772522463, US

(73) Assignee: SHELL OIL COMPANY, Houston, TX (US)

(21) Appl. No.: 11/794,447

(22) PCT Filed: Dec. 27, 2005
(86) PCT No.: PCT/EP2005/057174

§ 371 Date: Jan. 21, 2009

(30) Foreign Application Priority Data
Dec. 31, 2004 (EP) 04107076.4

(51) Int. Cl. C07D 301/03; B01D 53/22; B01J 19/00

(52) U.S. Cl. 549/523; 95/45; 422/187

(57) ABSTRACT

Integrated preparation and separation process comprising a preparation process wherein a byproduct is produced; and a gas separation process wherein a first component is separated from a mixture of components by diffusion of the first component through a porous partition into a stream of sweeping component; wherein the byproduct produced in the preparation process is subsequently used as the sweeping component in the separation process, and an industrial set-up for use in such a process.

### FIELD OF THE INVENTION

This invention relates to an integrated separation and preparation process.

### BACKGROUND OF THE INVENTION

In chemical industry several separation techniques are available to separate two or more components in a gaseous mixture. Examples of such separation techniques are known in the art and can be found in e.g. chapter 5.7 of “Process Design Principles” by W. Seider et al., published by John Wiley & Sons, inc. 1999.

The most generally applied technique is distillation. A disadvantage of distillation techniques, however, is the large amount of energy that is consumed to establish the separation of those compounds in a mixture.

Another technique that can be used is membrane separation by gas permeation. Herein a gas mixture is compressed to a high pressure and brought into contact with a non-porous membrane. The permeate passes the membrane and is discharged at a low pressure whereas the retentate does not pass through the membrane and is maintained at the high pressure of the feed. Examples for such a membrane separation method are described in U.S. Pat. No. 5,435,836 and U.S. Pat. No. 6,395,243. In these processes involving a gas separation via a membrane, in order to pass through the membrane, the gas molecules need to interact with the membrane. This however requires the application of a high pressure differential over the membrane between the retentate and the permeate side of the membrane. Due to the pressure differences required, such membrane techniques still require a considerable amount of energy and costly equipment for maintenance of the pressure differential, for instance by vacuum, or pressure pumps, even if a high sweep flow volume and highly selective membranes are employed.

U.S. Pat. No. 1,496,757, dating from 1924, describes a process of separation gases which comprises diffusing the gases through a diffusion partition, removing the diffused gas away from the partition by means of a sweeping material and removing the sweeping material from the diffused gas. The process is said to operate on the principle of repeated fractional diffusion. This process differs from separation processes involving membranes as described above in the fact that no or hardly any pressure differential is present, while the mass transfer is controlled by frictional diffusion with a sweep gas component continuously added to one chamber and diffusing counter-currently through the porous partitioning layer. This process thus does not require the use of expensive selectively permeable membranes.

Recently, M. Geboers, in his article “FricDiff: A novel concept for the separation of azeotropic mixtures”, OSPT Process Technology, PhD projects in miniposter form, published by the National Research School in Process Technology OSPT (2003) page 139, described a process for separating an azeotropic vapour mixture of 2-propanol (IPA) and water by letting it inter-diffuse with CO₂. In a subsequent step separation of the 2-propanol and CO₂ proceeds via condensation.

A disadvantage of this process is the required separation of product from the CO₂ stream, and if applied on an industrial scale, the procurement of a large sweep gas stream.

The use of the described diffusion-based separation method can thus still be improved by integration with a preparation process. The subject invention therefore provides for an integrated separation and preparation process.

### SUMMARY OF THE INVENTION

Accordingly, the present invention provides an integrated preparation and separation process comprising a preparation process wherein a byproduct is produced; and

a gas separation process wherein a first component is separated from a mixture of components by diffusion of the first component through a porous partition into a stream of sweeping component;
wherein the byproduct produced in the preparation process is subsequently used as the sweeping component in the separation process.

By using the byproduct of the preparation process as sweeping component in a subsequent separation process, more effective use of this byproduct is made and an advantageous integrated separation and preparation process is obtained. A “separate” sweeping component can be avoided, because a byproduct of the preparation process can be used as sweeping component.

The process according to the invention is especially advantageous in a process wherein the mixture of components from which the first component is separated is an azeotropic mixture, in view of the extensive costs of conventional distillation techniques for separation of such an azeotropic mixture.

The invention furthermore provides an industrial set-up in which the above process can be carried out.

### FIGURES

FIG. 1 is a schematic separation unit according to the invention.

FIG. 2: Schematic representation of the configuration of comparative example 1

FIG. 3: Schematic representation of the configuration of example 2

### DETAILED DESCRIPTION OF THE INVENTION

By an integrated separation and preparation process is understood a process wherein one or more of the components involved in the separation process is also a component involved in the preparation process. In the process of the present invention, the component used in the separation process as a sweeping component is used as a feed component in the preparation process.

By a gas separation process is understood that during this separation process at least part of the first component, mixture of components and sweeping component is in the gaseous state during the separation process. Preferably at least 50% wt of the first component, mixture of components and sweeping component is in the gaseous state, more preferably at least 80% wt, and even more preferably in the range from 90 to 100% wt is in the gaseous state. Most preferably all components are completely in a gaseous state during the separation process. A component which is normally in the liquid state under ambient temperature (25° C.) and pressure (1 bar) can be vaporized to the gaseous state, for example by increasing temperature or lowering pressure, before diffusing through the porous partition. The diffusion during the gas separation process is hence preferably gas diffusion.

Without wishing to be bound by any kind of theory, the diffusion of the first component through the porous partition during the separation process is thought to be based on the so-called principle of frictional diffusion. This frictional diffusion is believed to be due to a difference in rate of diffusion of a one component compared to one or more other components. As explained also in U.S. Pat. No. 1,496,757, a component having a faster rate of diffusion will more quickly pass a porous partition than a component having a slower rate of diffusion. The quicker component can be removed by the stream of sweeping component, resulting in a separation of such a first, quicker component from the remaining components. In the above a quicker component is understood to be a component having a higher binary diffusion coefficient together with the sweeping component than a slower component.

By a sweeping component is understood a component which is able to sweep away first component that has diffused through the porous partition. It can be any component known to the skilled person to be suitable for this purpose. Preferably a component is used which is at least partly gaseous at the temperature and pressure at which the separation process is carried out. More preferably a sweeping component is used which is nearly completely, and preferably completely gaseous at the temperature and pressure at which the separation process is carried out. For practical purposes the invention may frequently be carried whilst using a sweeping component having a boiling point at atmospheric pressure (1 bar) in the range from −200 to 500° C. More preferably a sweeping component is used sweeping component having a boiling point at atmospheric pressure (1 bar) in the range from −200 to 200° C. Examples of components that can be used as sweeping component include carbon monoxide, carbon dioxide, hydrogen, water, oxygen, oxides, nitrogen-containing compounds, alkanes, alkenes, alkanols, aromatics, ketones.

The mixture and the sweeping component are separated by a porous partition, through which the first component diffuses from the mixture into the stream of sweeping component.

The porous partition can be made of any porous material known to the skilled person to be suitable for use in a process where it is contacted with the reactants. The porous partition can be made of a porous material that assists in the separation of the components by for example adsorption or absorption effects, provided that the separation by diffusion prevails.

According to M Stanoevic, Review of membrane contactors designs and applications of different modules in industry, FME Transactions (2003) 31, 91-98, a membrane phase, which is set between two bulk phases, has the ability to control mass transfer between the two bulk phases in a membrane process. Contrary to such a membrane, the porous partitioning layer according to the subject invention is set between the two bulk phases, but has in principle no ability to control the mass transfer of any of the species involved. It does therefore essentially not interact with the species to be separated other than offering pores, but merely serves to avoid mixing of the two bulk phases, contrary to membrane separations.

The subject porous partition is thus essentially not a selectively permeable membrane. A membrane is a barrier that allows some compounds to pass through, while effectively hindering other compounds to pass through, thus a semi-permeable barrier of which the pass-through is determined by size or special nature of the compounds. Membranes used in gas separation techniques are for instance those disclosed in U.S. Pat. No. 5,843,209. Membranes selectively control mass transport between the phases or environments.

Contrary to such membranes, the porous partition is a barrier that allows the flow of all components, albeit at different relative rates of diffusion. Without wishing to be bound to any particular theory it is believed that in the porous partitioning, the mass transfer is controlled by frictional diffusion with a sweeping gas component continuously added to one chamber and leaving the other chamber and diffusing counter-currently through the porous partitioning layer.

Preferably the material used for the porous partition is essentially inert or inert to the components used in the separation process. In practice the invention may frequently be carried out whilst using filter cloth, metal, plastics, paper, sandbeds, zeolites, foams, or combinations thereof as material for the porous partition. Examples include expanded metals, e.g. expanded stainless steel, expanded copper, expanded iron; woven metals, e.g. woven copper, woven stainless steel; cotton, wool, linen; porous plastics, e.g. porous PP, PE or PS. In a preferred embodiment the porous partition is prepared from woven or expanded stainless steel.

The convective volumetric flow (m³/s) across the porous partition layer (assuming laminar or Poiseuille flow) is given by formula I:

$$Q = \frac{πΔ P ɛ {d}_{p}^{4}}{128 μδ} \qquad ( I )$$

wherein ε represents the porosity (fraction of surface area covered by pores), dₚ represents the pore diameter, δ represents the thickness of the porous layer, and ΔP represents the pressure drop across the porous layer as well as the physical properties of the gas (viscosity and density).

Preferred porous material should have a high porosity (ε) to maximise the useful surface area. The preferred porous layers porous have a porosity of more than 0.5, preferably more than 0.9, yet more preferably more than 0.93.

The thickness of the porous layer is preferably as low as possible. Without wishing to be bound to any particular theory, it is believed that the diffusive rate is inversely proportional to the thickness of the porous layer, and thus the required surface area of the porous layer is proportional to the thickness.

The porous partition can vary widely in thickness and may for example vary from a partition having a thickness of 1 or more meters to a partition having a thickness of 1 or more nanometres. For practical purposes the invention may frequently be carried out using a porous partition having a thickness in the range from 0.0001 to 1000 millimetres, more preferably in the range from 0.01 to 100 millimetres, and still more preferably in the range from 0.1 to 10 millimetres. Preferred porous layers have a thickness in the range of from 0.5 to 1.5 millimetres, preferably in the range of from 0.8 to 1.2 millimetres, and more preferably in the range of from 0.9 to 1.1 millimetres.

The amount, size and shape of the pores used in the porous partition may vary widely. The shape of the pores used in the porous partition may be any shape known to the skilled person to be suitable for such a purpose. The pores can for example have a cross-section shaped as slits, squares, ovals or circles. Or the cross-section may have an irregular shape. For practical purposes the invention may frequently be carried out using pores having a cross-section in the shape of circles. The diameter of cross-section of the pores may vary widely. It is furthermore not necessary for all the pores to have the same diameter. For practical purposes the invention may frequently be carried out using pores having a cross-section “shortest” diameter in the range from 1 manometer to 10 millimetre. By the “shortest” diameter is understood the shortest distance within the cross-section of the pore. Preferably this diameter lies in the range from 20 nanometre to 2 millimetres, more preferably from 0.1 to 1000 micrometer, more preferably in the range from 10 to 100 micrometer.

Preferably, the pores (dₚ) in the material should be relatively small to prevent convective flow. The exact size and proportions depend on the thickness of the porous layer (Δ) and the pressure drop (ΔP) across the porous layer as well as the physical properties of the gas (viscosity and density).

Pores having a small diameter, e.g. in the range from 0.1 to 100 nanometres have the advantage that the control on pressure differences becomes more easy. Pores having a larger diameter, e.g. in the range from 100 to 1000 nanometres have the advantage that a better separation can be obtained. For instance at a pressure drop (ΔP) of around 10 Pa across the porous partition, the pores should have a diameter below 10 micrometer to prevent substantial convective flow as compared to the desired diffusive flow. At a pressure drop (ΔP) of 1 Pa, pores having a diameter of 30 micron should be preferred. However, pressure drop and pore diameter should be chosen in such way that a Knudsen diffusion regime is avoided.

Is it understood that the relative rates of diffusion through the porous layer of different gases are dependent on the relative magnitudes of their binary diffusion coefficients, and not or only to a lesser extent on the properties of the porous material.

The pores may furthermore vary widely in tortuosity, that is, they may vary widely in degree of crookedness. Preferably however, the pores are straight or essentially straight and have a tortuosity in the range from 1 to 5, more preferably in the range from 1 to 3.

The number of pores used in the porous partition may also vary widely. Preferably 1.0-99.9% of the total area of the porous partition is pore area, more preferably 40 to 99%, and even more preferably 70 to 95% of the total area of the partition is pore area. By pore area is understood the total surface area of the pores. For practical purposes the invention may frequently be carried out using a number of pores and a pore size such that the ratio of total surface area of pores in the partition to the gas volume of the mixture of components lies in the range from 0.01 to 100,000 m²/m³, preferably in the range from 1 to 1000 m²/m³.

The length of the porous partition in the direction of the flow of the stream of sweeping component may also vary widely. When the length of the layer is increased both building costs of the separation as well as the extent of separation increase. For practical purposes the invention may frequently be carried out using a porous partition having a length along the flow-direction of the sweeping component in the range from 0.01 to 500 meters, more preferably in the range from 0.1 to 10 meters.

The residence time of the sweeping component and/or the mixture of components in the separation unit can vary widely. For practical purposes the invention may frequently be carried out using a residence time for sweeping component and/or the mixture of components in the separation unit in the range from 1 minute to 5 hour. Preferably a residence time is used in the range from 0.5 to 1.5 hours.

The residence time of the sweeping component and/or the mixture of components in the separation unit can vary widely. For practical purposes the invention may frequently be carried out using a residence time for sweeping component and/or the mixture of components in the separation unit in the range from 1 minute to 5 hour. Preferably a residence time is used in the range from 0.5 to 1.5 hours.

The velocity of the sweeping component used in the process of the invention may vary widely. For practical purposes the invention may frequently be carried out at a velocity of the sweeping component in the range from 1 to 10,000 meters/hour, preferably in the range from 3 to 3000 meters/hour and more preferably in the range from 10 to 1000 meters/hour. If not stationary, similar velocities can be used for the mixture of components.

The flux of the diffusion of the first component through the porous partition can vary widely. For practical purposes the invention may frequently be carried out at a diffusion flux of the first component through the porous partition in the range from 0.03 to 30 kg/m²/hour, preferably in the range from 0.1 to 10 kg/m²/hour and more preferably in the range from 0.5 to 1.5 kg/m²/hr.

For practical purposes the invention may frequently be carried out by removing from 10 to 100% wt of the first component, based on the total amount of first component present in the mixture of components when starting the separation process, from the mixture of components. More preferably at least 30% wt, and more preferably at least 50% wt of first component present in the mixture is removed from the mixture of components during the separation process. Even more preferably in the range from 70 to 100% wt of first component, based on the total amount of first component present in the mixture of components when starting the separation process, is removed from the mixture of components during the separation process. Especially when removing a high percentage, e.g. in the range from 70 to 100% wt, of first component from the mixture of components, other components might also diffuse from the mixture of components into the stream of sweeping component. When such other components co-diffuse, they can be removed in an additional intermediate step before entering the preparation process; or, alternatively, such other co-diffused components can remain in admixture with the sweeping component and/or with the diffused first component during a subsequent preparation process. Possibly such other co-diffused components can removed via a bleed stream in such a subsequent preparation process.

In another embodiment the separation process according to the invention can be combined with an additional separation process, including conventional distillation and/or membrane separation. The additional separation process can for example be used for removing other co-diffused components from the mixture of sweeping component and first component. Or it can be used to remove other components from the mixture of components, before or after removal of the first component. Furthermore an additional separation process can be used to further remove first component from a mixture of components from which at least part of the first component has already been removed.

The first component can be separated from a stationary mixture by diffusion through a porous partition into a stream of sweeping component. Preferably, however, a separation process is used, wherein the first component is separated from a stream of a mixture of components on one side of a porous partition, by diffusion through such porous partition, into a stream of sweeping component on the other side of the porous partition. Such a separation process might be carried out co-currently or counter-currently. Preferably, however, such a separation process is carried out whilst having a stream of the mixture of components and a stream of sweeping component flowing counter-currently in respect of each other. The separation process can be carried out continuously, semi-batch or batch-wise. Preferably the separation process is carried out continuously.

The flow velocity of the stream of sweeping component can vary widely. For practical purposes the invention may frequently be carried out using a flow velocity for the stream of sweeping component in the range from 0.01 to 300 kmol/hour, more preferably in the range from 0.1 to 100 kmol/hour. The flow velocity of any flow of mixture of components (if not stationary) can also vary widely. For practical purposes the invention may frequently be carried out using a flow velocity for the stream of sweeping component in the range from 0.01 to 300 kmol/hour, more preferably in the range from 0.1 to 100 kmol/hour.

The temperature applied during the separation process can vary widely. Preferably such a temperature is chosen that all components are completely gaseous during the diffusion process. More preferably the temperature in the separation process is the same to the temperature in the preparation process. For practical purposes the invention may frequently be carried out using a temperature in the range from 0 to 500° C., preferably in the range from 0 to 250° C. and more preferably in the range from 15 to 200° C.

The pressures applied may vary widely. Preferably such a pressure is chosen that all components are completely gaseous during the diffusion process. More preferably the pressure in the separation process is the same to the pressure in the preparation process. For practical purposes the invention may frequently be carried out using a pressure in the range from 0.01 to 200 bar (1×10³ to 200×10⁵ Pa), preferably in the range 0.1 to 50 bar. For example the separation process can be carried out at atmospheric (1 atm., i.e. 1.01325 bar) pressure.

Independently from the overall pressures applied, the pressure difference over the porous partition is maintained as small as possible, i.e. the pressure on both sides of the porous partition is essentially equal, e.g. in the range of 0.0001 to 0.1 bar, provided that separation by diffusion prevails over any separation due to mass motion because of large pressure differences. The pressure difference preferably is in the range of from 0.0001 to 0.01 bar, more preferably in the range of 0.0001 to 0.001 bar, yet more preferably in the range 0.0001 to 0.0001 bar, and most preferably in the range of from 0.0001 to 0.0005 bar. Hence, the pressure on both sides of the porous partition is considered nearly equal or essentially equal.

This may preferably be achieved by adding a pressure balancing means into the system, for instance by providing a flexible diaphragm that allows to pass on pressure peaks in one of the two fluid streams to the other.

The separation process can be carried out in any apparatus known to the skilled person to be suitable for this purpose. For example separation units can be used such as the ones exemplified in U.S. Pat. No. 1,496,757. Preferably a separation unit, suitable for separating a first component from a mixture of components by diffusion of the first component through a porous partition into a stream of sweeping component, is used which separation unit comprises

a first chamber;

a second chamber, separated from the first chamber by a porous partition;

a first inlet for conveying a mixture of components to the first chamber;

a first outlet for discharging the remainder of the mixture of components after at least part of the first component has been removed from the first chamber;

a second inlet for conveying a sweeping component into the second chamber;

a second outlet for discharging a mixture of sweeping component and diffused first component from the second chamber.

The first and second chamber can be arranged in several ways. In a preferred embodiment one chamber is formed by the inside space of a tube and the other chamber is formed by a, preferably annular, space surrounding such tube.

Such an embodiment is considered to be novel and hence the present invention further provides a separation unit, suitable for separating a first component from a mixture of components by diffusion of the first component through a porous partition into a stream of sweeping component, which separation unit comprises

an outer tube; and

an inner tube, which inner tube has a porous wall, and which inner tube is arranged within the outer tube, such that a first space is present within the inner tube and a second space is present between the outer surface of the inner tube and the inner surface of the outer tube; and

a first inlet for conveying fluid into the first space and

a first outlet for discharging fluid from the first space; and

a second inlet for conveying fluid into the second space and

a second outlet for discharging fluid from the second space.

The fluids are, each independently, for preferably at least 50% wt in the gaseous state, more preferably at least 80% wt, and even more preferably in the range from 90 to 100% wt. Most preferably the fluids are nearly completely or completely gaseous.

Furthermore the inner tube and the outer tube are preferably arranged essentially co-axially. The first space can either be used as a first chamber or as a second chamber and the second space can respectively be used as a second chamber or as a first chamber. Both the first as well as the second space can have multiple inlets and outlets. Preferably the first space present within the inner tube has only one inlet and only one outlet. The second space preferably has two or more, preferably 2 to 100 inlets and/or outlets or an inlet and/or outlet in the shape of a circular slit.

The inner tube can be arranged substantially eccentrically within the outer tube such that the central axis of the inner tube is arranged substantially parallel to the central axis of the outer tube. Preferably, however the inner tube is arranged substantially concentrically within the outer tube such that the central axis of the inner tube substantially coincides with the central axis of the outer tube.

The cross-section of the tubes can have any shape known to the skilled person to be suitable. For example, the tubes can independently of each other have a cross-section in the shape of a square, rectangle, circle or oval. Preferably the cross-section of the tubes is essentially circular.

In a different preferred embodiment, the first and the second chamber are separated by a porous partition formed by stacks of plates or sheets of the porous material. In these stacks, at least two plates, i.e. an upper plate and a lower plate comprising the porous partition material are layered above each other in such way as to provide an intermediate compartment, which is blocked off at one end, while fluidly connected to an open space at the other end. In stacks comprising more than two layers, the openings on adjacent sides of each intermediate compartment are blocked alternately. Hence, the stack separates a first chamber and a second chamber as set out above, while the chambers are at least in part formed by the stack. The plates of comprising the porous partition material may be at any suitable shape, for instance rectangular; they may be of even shape and size, or uneven. The latter is preferred since then one side of a plate is longer than the other side, and thus the flow of the faster flowing gas passes across the shorter distance, thereby lowering the pressure drop.

The compartments are typically defined by spacers or structures that are offset and support the porous partition. The spacer, along with the porous partition material connected thereto defines the intermediate compartment which may serves as retentate or sweeping compartment. The pressure drop may also conveniently be adjusted by using different spacers for the sweep gas and feed gas compartments.

Adjacent compartments have the porous partition positioned there-between in the shape of layered plate-like or sheet-like structures, thereby providing a flow path for both fluid streams with a large surface. The assembly of retentate and sweeping compartments may be in alternating order or in any of various arrangements necessary to satisfy design and performance requirements. The stack arrangement is typically bordered by a seal at one end and a fluid connection to another compartment at an opposite end.

The compartments are suitably placed into a separator vessel such that they are fluidly connected either to a fluid stream, while they are sealed towards the respective opposite fluid stream, thus separating the two fluid feed streams. The feeds of the two fluid streams are fed preferably in a cross flow arrangement to the alternate sides of the separator vessel, i.e. to arrive at perpendicular flow or cross-flow direction towards each other. This serves to bring the flows out of line (i.e. not co-linear flows) so that they can be fed to the vessels fluid inlet and outlet openings more easily.

The separation device suitable comprises a vessel comprising a first fluid inlet opening positioned proximate to a side of the vessel and a first fluid outlet opening positioned proximate to an opposing side of the vessel; a second fluid inlet opening positioned proximate to a side of the vessel and a second fluid outlet opening positioned proximate to an opposing side of the vessel, wherein the first and second inlets and outlets respectively are position in such way, that the flow direction of a first fluid stream entering the vessel at the first inlet, and leaving it at the first outlet, and a second fluid stream entering the vessel at the second inlet, and leaving it at the second outlet are essentially perpendicular to each other; and wherein the porous partition between the two fluids comprises a stack of plate-like structures which are sealed toward the first fluid stream, while fluidly connected to the second fluid stream, thereby forming an exterior flow space for the first stream defined at least partially by and positioned at least partially between an upper plate and a lower plate of porous material, and an interior flow space for the second stream, defined at least partially by and positioned at least partially between the opposite sides of the upper plate and the lower plate to prevent fluid flow from the exterior flow space into the interior flow space. The advantage of using a stacked separation device is that in cross-flow many parallel compartments are alternately connected to the feed stream and to the sweep gas stream, thus providing for a large surface in a relatively compact arrangement.

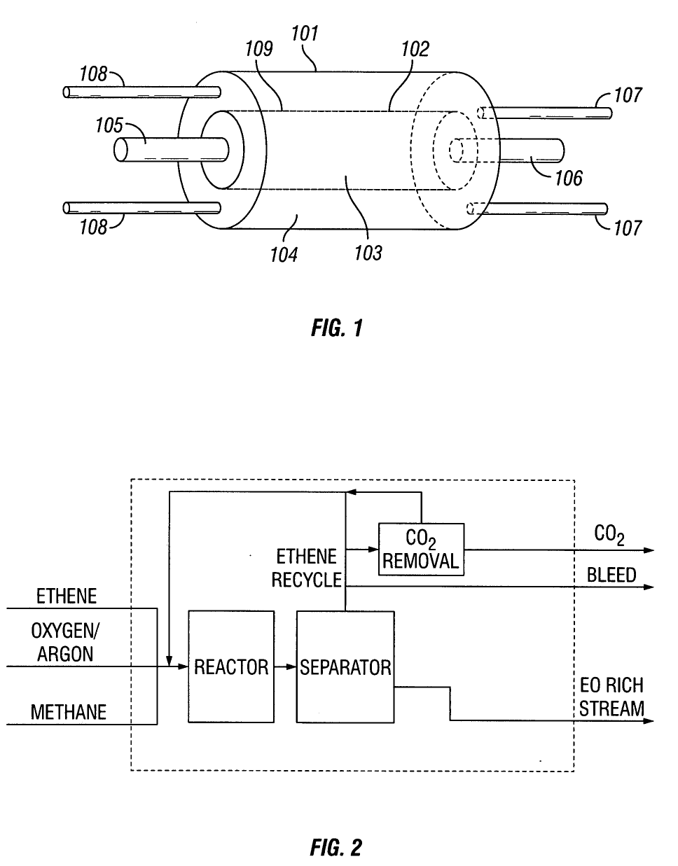

The invention will be described by way of example with reference to FIG. 1. FIG. 1 is a schematic three-dimensional view of a separation unit according to the present invention. FIG. 1 illustrates a separation unit having an outer tube (101) and an inner tube (102), which inner tube is co-axially arranged within the outer tube, such that

a first space (103) is present within the inner tube (102) and
a second space (104) is present between the outer surface of the inner tube (102) and the inner surface of the outer tube (101); and
comprising an inlet (105) into the first space and an outlet (106) from the first space; and an inlet (107) into the second space and an outlet (108) from the second space;
which inner tube has a porous wall (109).

In a further preferred embodiment the separation process is carried out in a separation device comprising a multiple of separation units. Preferably in the range from 2 to 100,000, more preferably in the range from 100 to 10,000 separation units per separation device. Such a separation device is considered to be novel and therefore the present invention furthermore provides a separation device comprising two or more separation units, suitable for separating a first component from a mixture of components by diffusion of the first component through a porous partition into a stream of sweeping component, wherein each separation unit can comprise

a first chamber;

a second chamber, separated from the first chamber by a porous partition;

a first inlet for conveying a mixture of components to the first chamber;

a first outlet for discharging the remainder of the mixture of components after at least part of the first component has been removed from the first chamber;

a second inlet for conveying a sweeping component into the second chamber;

a second outlet for discharging a mixture of sweeping component and diffused first component from the second chamber.

The separation units can be arranged in the separation device in any manner known to suitable for this purpose by the skilled person. Preferably the separation units are arranged sequentially or parallel to each other in the separation device. The separation units can for example be sequentially arranged in an array. If such an array of sequentially arranged separation units is used, any pressure loss on either one side is preferably compensated by a intermediate stream of respectively mixture of components or sweeping component.

In an advantageous embodiment, the first or second chambers of two or more separation units are blended together such that two or more separation units share the same first or second chamber.

For example the present invention provides a multitubular separation device comprising

a substantially vertically extending vessel,

a plurality of tubes having a porous wall, arranged in the vessel parallel to its central longitudinal axis of which the upper ends of the tubes are fixed to an upper tube plate and in fluid communication with a top fluid chamber above the upper tube plate and of which the lower ends are fixed to a lower tube plate and in fluid communication with a bottom fluid chamber below the lower tube plate,

supply means for supplying a first fluid to the top fluid chamber and

an effluent outlet arranged in the bottom fluid chamber;

supply means for supplying a second fluid to the space between the upper tube plate, the lower tube plate, the outer surface of the tubes and the vessel wall and

an effluent outlet from such space between the outer surface of the tubes and the vessel wall.

The fluids are, each independently, for preferably at least 50% wt in the gaseous state, more preferably at least 80% wt, and even more preferably in the range from 90 to 100% wt. Most preferably the fluids are nearly completely or completely gaseous.

A mixture of components can for example be supplied to the space inside the tubes or to the space between the outer surface of the tubes and the inner surface of the vessel wall; and the sweeping gas can be supplied to respectively the space between the outer surface of the tubes and the inner surface of the vessel wall or the space inside the tubes.

In the preparation process one or more reactants can be reacted to one or more products and at least one byproduct. By reacting is understood that the sweeping component is chemically changed. For example, the sweeping component can be chemically split into two or more separate products or the sweeping component can be reacted with one or more other components into one or more products. Examples of possible reactions include but are not limited to hydration, dehydration, hydrogenation and dehydrogenation, oxygenation, hydrolysis, esterification, amination, carbonation, carbonylation, carboxylation, desulfurisation, deamination, condensation, addition, polymerisation, substitution, elimination, rearrangement, disproportionation, acid-base, telomerisation, isomerisation, halogenation, dehalogenation and nitration reactions. The reaction conditions applied can vary widely and can be those known to the skilled person to be suitable for such reaction. In practice, the invention may frequently be carried out at a temperature in the range from −100 to 500° C., more preferably in the range from 0 to 300° C., and at a pressure in the range of 0.0001 to 100 bars, more preferably in the range of 1 to 50 bars. Any type of reactor known by the skilled person to be suitable for a reaction can be used. Examples of types of reactors include a continuously stirred reactor, slurry reactor or tube reactor.

One or more of reactions in the preparation process can optionally be carried out in the presence of a catalyst. Any catalyst known to the skilled person to be suitable for a specific reaction applied can be used. Such a catalyst can be homogeneous or heterogeneous and might for example be present in solution, slurry or in a fixed bed. The catalyst can be removed in a separate unit.

The steps in the process of the invention can each be carried out in a continuous, semi-batch or batch manner. For example the separation process can be carried out in a continuous or semi-batch manner whereas the subsequent preparation process can be carried out in a batch manner. In a preferred embodiment, all steps are carried out in a continuous manner. Hence the present invention also provides a process according to the invention wherein this process is continuous.

The integrated separation and preparation process is preferably carried out in an industrial set-up comprising

a separation device comprising one or more separation units suitable for separating a first component from a mixture of components by diffusion of the first component through a porous partition into a stream of sweeping component, comprising one or more first chambers, one or more second chambers, separated from the first chamber or chambers by a porous partition, one or more inlets and one or more outlets,

one or more reactors comprising one or more inlets and one or more outlets, of which at least one outlet comprises a stream of byproduct of a reaction, wherein such byproduct outlet of one or more preparation units is connected directly or indirectly to one or more inlets of one or more separation units.

In a preferred embodiment the integrated preparation and separation process comprises the steps of

a) reacting one or more reactants to obtain a reaction mixture comprising a product, at least one byproduct and at least one contaminant;
b) separating the byproduct from the reaction mixture, to obtain byproduct and a mixture comprising product and at least one contaminant;
c) gas separating the contaminant from the mixture of product and contaminant by diffusion of the contaminant through a porous partition into a stream of byproduct, to obtain a stream of mixture of contaminant and byproduct and a stream of purified product.

Optionally the product obtained in step c) can be purified further by for example distillation methods.

In such a process step a) can be carried out as described hereinabove for the preparation process and step c) can be carried out as described herein above for the separation process.

The separation in step b) can be carried out in any manner known by the skilled person to be suitable for this purpose. Preferred methods include gas-liquid separation and distillation.

In a further preferred embodiment the integrated preparation and separation process comprises the steps of

a) reacting one or more reactants to obtain a reaction mixture comprising a product, at least one unreacted reactant, at least one byproduct and at least one contaminant;
b) separating the reaction mixture, to obtain a stream of byproduct, a stream of a mixture of unreacted reactant and contaminant and a stream of purified product;
c) gas separating the contaminant from at least part of the mixture of unreacted reactant and contaminant by diffusion of the contaminant through a porous partition into a stream of byproduct, to obtain a stream of mixture of contaminant and byproduct and a stream of purified unreacted reactant.

Optionally the product obtained in step b) and the unreacted reactant in step c) can be purified further by for example distillation methods.

In such a process step a) can be carried out as described hereinabove for the preparation process and step c) can be carried out as described herein above for the separation process.

A process as described above, wherein an unreacted reactant can be purified, can advantageously be used in an integrated preparation and separation process wherein unreacted reactant is recirculated to the reaction. Conventionally contaminants in such an unreacted reactant recirculation stream are removed by a bleed stream. By removing contaminant from this unreacted reactant with the process according to the invention, an advantageously smaller bleed stream can be used. The stream of unreacted reactant and contaminant entering the separation unit can be part of the mixture of unreacted reactant and contaminant obtained in step b) or can be the whole of the mixture obtained in step b). When only part of the mixture of unreacted reactant and contaminant obtained in step b) is separated in step c), this part is preferably in the range from 0.1 to 50% wt, more preferably in the range from 0.1 to 30% wt of the total mixture separated in step b). Using only part of the mixture of unreacted reactant and contaminant obtained in step b) in step c) has the advantage that a small separation unit can be used. Such a small separation unit can easily be incorporated in an already existing industrial set-up. Using the total of the mixture of unreacted reactant and contaminant obtained in step b) in step c) has the advantage that more contaminant can be removed and that the bleed stream can be further reduced.

The process of the present invention is widely applicable. For example, the process can be used in a process for the preparation of an alkylene oxide, wherein alkene is reacted with oxygen which oxygen is contaminated with an inert gas, and in which process carbon dioxide is prepared as a byproduct.

The present invention therefore also provides a process for the preparation of an alkylene oxide comprising the steps of

a) reacting an alkene with oxygen, which oxygen is contaminated with an inert gas, to obtain a reaction mixture comprising an alkylene oxide, unreacted alkene, carbon dioxide and inert gas;
b) separating the reaction mixture, to obtain a stream of carbon dioxide, a stream of a mixture of alkene and inert gas and a stream of purified alkylene oxide;
c) gas separating inert gas from at least part of the mixture of unreacted alkene and inert gas by diffusion of the inert gas through a porous partition into a stream of carbon dioxide, to obtain a stream of mixture of inert gas and carbon dioxide and a stream of purified alkene.

Preferably the alkene is an alkene having from 2 to 10 carbon atoms. Preferred examples include ethene, propene, butenes and pentenes. Preferably the process of the invention is used to prepare a corresponding alkylene oxide having from 2 to 10 carbon atoms. Preferred examples include ethylene oxide, propylene oxide, butylene oxides and pentylene oxides. In addition a mixture of alkenes can be converted into a mixture of corresponding alkylene oxides. Most preferred is a process wherein ethene or propene or a mixture thereof is converted into ethylene oxide, propylene oxide or a mixture thereof. If desirable, in addition to the above, a further diluent gas can be used such as for example methane. Such a diluent gas can be used as exemplified for example in U.S. Pat. No. 5,519,152. In addition to the above furthermore a homogeneous or heterogeneous catalyst can be used in the process.

Carbon dioxide can be prepared in such a process as a byproduct. The inert gas can for example be nitrogen or argon. Preferably the inert gas is argon.

In another process an alkane can be used as sweeping gas, the present invention therefore also provides a process comprising gas separating hydrogen from a mixture of hydrogen, methane and carbon dioxide by diffusion of the hydrogen through a porous partition into a stream of alkane, to obtain a mixture of alkane and hydrogen and a purified mixture of methane and carbon dioxide.

Preferably such an alkane has 2 to 10 carbon atoms. Preferred examples of such an alkane include ethane, propane and butanes.

The invention will be illustrated by the following non-limiting examples.

### COMPARATIVE EXAMPLE 1

In a comparative example ethylene oxide (EO) is prepared by reacting ethene with oxygen, which oxygen is contaminated with argon. After reaction, the reaction mixture is conveyed to a separator, where the mixture is separated by using a difference in absorption, wherein the reaction mixture is separated into an ethylene oxide rich stream and an ethene recycle stream. The ethene recycle stream further comprises the byproduct carbon dioxide and the contaminant argon. A bleed stream leaves the process to avoid the accumulation of argon. The process is illustrated by FIG. 2.

The process was modelled via a computer model. In this computer model the streams in the process are arranged such that bleed stream leaving the process comprises 3.5 kmol/hr of argon. In this case the bleed stream was found to further comprise 8.5 kmol/hr of ethene. Methane is added to the process as a diluent. The process was modelled via a computer model, resulting in a bleed stream as summarized in table 1:

TABLE 1
Composition of bleed stream in comparative
example 1 illustrated in FIG. 2.

| Component | Mole percentage (%) | Mole flow (kmol/h) |
|---|---|---|
| CO₂ | 6.25 | 2.5 |
| Ar | 8.75 | 3.5 |
| C₂H₄ | 21.25 | 8.5 |
| CH₄ | 63.75 | 25.5 |

### EXAMPLE 2

In an example according to the process of the invention the preparation of ethylene oxide is carried out as described above for example 1, except that the bleed stream is further separated in a gas separation unit according to the invention, wherein argon is removed by diffusion through a porous partition into a CO₂ stream.

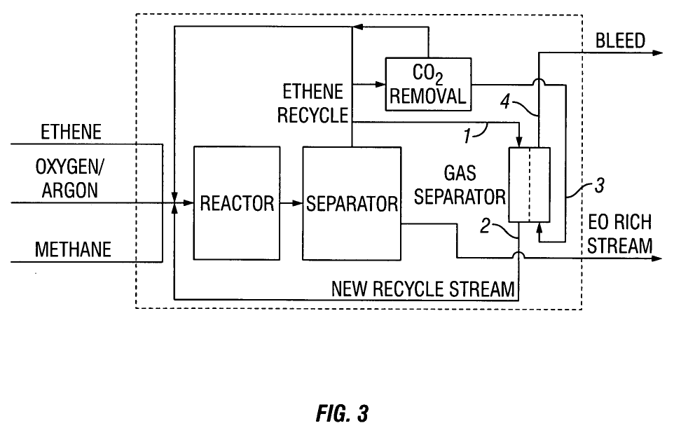

The process is illustrated by FIG. 3.

The process was modelled via a computer model. In this computer model, the gas separation was carried out at a temperature of about 35° C. and at a pressure of about 1 bar; the gas separator had a length of 1.53 m and a porous partition thickness of 1 mm; and the total surface area of pores in the porous partition to gas volume was 100 m²/m³. The CO₂ stream that left the process in comparative example 1 (pure, 450 kmol/h), is now fed counter-currently to the argon containing bleed stream. The result is a new bleed stream (4), and a new recycle stream (2) with a lower argon concentration, than the former bleed stream (1). The streams are arranged such that the new bleed stream (4) leaving the process whilst comprises 3.5 kmol/hr of argon. In that case the new bleed stream comprises 7.8 kmol/hr of ethene as illustrated in table 2. The flows for the remaining streams as indicated in FIG. 3 are summarized in table 3 and 4.

TABLE 2
Composition of the new bleed stream (4) in example 2

| Component | Mole percentage (%) | Mole flow (kmol/h) |
|---|---|---|
| CO2 | 6.25 | 411.9 |
| Ar | 8.75 | 3.5 |
| C₂H₄ | 21.25 | 7.8 |
| CH₄ | 63.75 | 26.8 |

TABLE 3
Flows as indicated in FIG. 3 for example 2

| | | | | | Ratio |
|---|---|---|---|---|---|
| | F_{CO2} | F_{Ar} | F_{C2H4} | F_{CH4} | F_{C2H4}/ |
| Stream no. | (kmol/h) | (kmol/h) | (kmol/h) | (kmol/h) | F_{Ar} (-) |
| 1 | 3.00 | 4.20 | 10.20 | 30.60 | 2.43 |
| 2 | 41.10 | 0.70 | 2.43 | 3.77 | 3.49 |
| 3 | 450.00 | — | — | — | — |
| 4 | 411.90 | 3.50 | 7.77 | 26.83 | 2.22 |

TABLE 4
Composition new recycle stream (2) for example 2

| Component | Mole percentage (%) | Mole flow (kmol/h) |
|---|---|---|
| CO₂ | 86 | 41.10 |
| Ar | 1 | 0.70 |
| C₂H₄ | 5 | 2.43 |
| CH₄ | 8 | 3.77 |

It can be seen that by using the gas separation according to the invention, it is possible to reduce the loss of ethene in the bleed stream from 8.5 to 7.8 kmol/h.

1. An integrated preparation and separation process comprising
a preparation process wherein a byproduct is produced; and
a gas separation process wherein a first component is separated from a mixture of components by diffusion of the first component through a porous partition into a stream of sweeping component;
wherein the byproduct produced in the preparation process is subsequently used as the sweeping component in the separation process.

2. The process of claim 1, wherein the pressure on both sides of the porous partition is essentially equal.

3. The process of claim 1 or, comprising the steps of
a) reacting one or more reactants to obtain a reaction mixture comprising a product, at least one byproduct and at least one contaminant;
b) separating the byproduct from the reaction mixture to obtain byproduct and a mixture comprising product and at least one contaminant; and
c) gas separating the contaminant from the mixture of product and contaminant by diffusion of the contaminant through a porous partition into a stream of byproduct to obtain a stream of mixture of contaminant and byproduct and a stream of purified product.

4. The process of claim 1, comprising the steps of
a) reacting one or more reactants to obtain a reaction mixture comprising a product, at least one unreacted reactant, at least one byproduct and at least one contaminant;
b) separating the reaction mixture to obtain a stream of byproduct, a stream of a mixture of unreacted reactant and contaminant and a stream of purified product; and
c) gas separating the contaminant from at least part of the mixture of unreacted reactant and contaminant by diffusion of the contaminant through a porous partition into a stream of byproduct to obtain stream of a mixture of contaminant and byproduct and a stream of purified unreacted reactant.

5. A process comprising the steps of
a) reacting an alkene with oxygen, which oxygen is contaminated with an inert gas, to obtain a reaction mixture comprising an alkylene oxide, unreacted alkene, carbon dioxide and inert gas;
b) separating the reaction mixture to obtain a stream of carbon dioxide, a stream of a mixture of alkene and inert gas and a stream of purified alkylene oxide; and
c) gas separating the inert gas from at least part of the mixture of unreacted alkene and inert gas by diffusion of the inert gas through a porous partition into a stream of carbon dioxide to obtain a stream of mixture of inert gas and carbon dioxide and a stream of purified alkene.

6. An industrial apparatus comprising
a separation device comprising one or more separation units suitable for separating a first component from a mixture of components by diffusion of the first component through a porous partition into a stream of sweeping component, said separation device comprising one or more first chambers, one or more second chambers separated from the first chamber or chambers by a porous partition, one or more inlets and one or more outlets; and
one or more reactors comprising one or more inlets and one or more outlets, of which at least one outlet may contain a stream of byproduct of a reaction, wherein such byproduct outlet is connected directly or indirectly to one or more inlets of one or more separation units.
